(19) United States
(12) Patent Application Publication
(10) Pub. No.: US 2025/0051812 A1
(43) Pub. Date: Feb. 13, 2025

(54) PRODUCTION OF CLEAN STARCH HYDROLYSATES

(71) Applicant: DANISCO US INC., Palo Alto, CA (US)

(72) Inventors: Robert I Christensen, El Sobrante, CA (US); Jayarama K Shetty, Pleasanton, CA (US)

(21) Appl. No.: 18/719,937

(22) PCT Filed: Dec. 16, 2022
(86) PCT No.: PCT/US2022/081777

§ 371 Date: Jun. 14, 2024

Related U.S. Application Data
(60) Provisional application No. 63/290,091, filed on Dec. 16, 2021.

(51) Int. Cl. C12P 19/02; C12N 9/26; C12P 7/10; C12P 19/14

(52) U.S. Cl. CPC C12P 19/02; C12N 9/2414; C12P 7/10; C12P 19/14

(57) ABSTRACT

Described is a process for producing high quality starch hydrolysates using course-ground grains and seeds in a high temperature steeping process. The process is ideal for producing high quality starch hydrolysates in an energy-efficient manner, as well as for producing high quality animal feed and oil co-products.

### CROSS REFERENCE TO RELATED APPLICATIONS

This application claims the benefit of U.S. Application No. 63/290,091, filed Dec. 16, 2021, which is hereby incorporated by reference in its entirety,

### FIELD OF THE INVENTION

Disclosed is a process for producing high quality starch hydrolysates using course-ground grains and seeds in an elevated temperature steeping process. The process is ideal for producing high quality starch hydrolysates in an energy-efficient manner, as well as for producing high quality animal feed co-products and oil.

### BACKGROUND

Wet milling and dry grind milling are the predominant processes/methods for processing grains into syrups, ethanol, other biochemicals and co-products, such as animal feed and oil. Corn is the predominant grain processed by these processes/methods, but wheat, barley, rye, sorghum, milo, rice and the like can be processed in a similar manner.

Using corn as an example, the wet-milling process is designed to extract maximum value from each component in a corn kernel. Corn kernels are first steeped in water at about 50° C. for about 36 hours with the optional addition of SO₂ and, optionally, lactic acid. Steeping breaks disulfide bonds and weakens the gluten starch matrix, promoting the release of starch granules. SO₂ also provides microbial control. Acid protease can be used in place of SO₂. The resulting steep water contains corn soluble solids such as sugars, salts, amino acids, peptides, proteins, and other micronutrients for potential fermentation organisms, but virtually no starch.

The softened kernels are then mechanically processed to remove the germ, which is further processed to recover high-value corn oil. Following germ removal, the remaining kernel components are screened to recover the fiber used to produce gluten feed for animals. Starch and some gluten pass through the screens and are subsequently subjected to centrifugation to separate lighter gluten protein (which can be added to the gluten meal) from starch. The high-quality starch can further be dried to produce dry starch and/or is subjected to enzymatic hydrolysis to produce hydrolyzed starch syrup which can then be converted to glucose, maltose, or other sugars, e.g., for the production of syrups. While such sugars can also be used as a fermentation feed stock for producing ethanol and other valuable biochemicals, they are more often used for valuable food products, including specialty syrups and high-fructose corn sugar.

The corn dry grind milling process is less capital-intensive process and focuses primarily on the production of grain ethanol. In this process, corn kernels are first milled to a medium-to-fine grind prior to enzymatic processing. Hammer milling is most common. Enzymatic processing occurs first with a thermostable α-amylase (optionally with protease and other enzymes) at or above the starch gelatinization temperature. Processing at higher temperatures requires more energy and higher costs. As with wet milling, the resulting maltodextrins can then be converted to glucose, or theoretically other sugars. However, dry grind milling tends to be a process using whole ground corn for industrial grade products and is not suitable for the production of food grade products. Accordingly, fuel ethanol, dried distillers' grains (a low-value animal feed product) and oil (for the production of biodiesel), tend to be the only products.

The need exists for superior ways to process grains to extract maximum value with minimal capital investment.

### SUMMARY

Described is a process for producing high quality starch hydrolysates using coarse-ground grains and seeds in an elevated temperature steeping process, followed by separation of the steep water from insoluble matter. The process is ideal for producing high quality starch hydrolysate in an energy-efficient manner, as well as for producing high quality animal feed products and oil. Aspects and embodiments of the present compositions and processes/methods) are summarized in the following, separately-numbered-paragraphs:

1. In one aspect, a process for producing a soluble starch hydrolysate from grains or seeds is provided, comprising steeping coarsely-ground grains or seeds at a temperature above the gelatinization temperature of starch in the grains or seeds in the presence of a thermostable α-amylase to produce a starch hydrolysate from starch present in the grains or seeds; and recovering the soluble starch hydrolysate from the steeping water.

2. In some embodiments of the process of paragraph 1, coarsely ground grains or seeds are substantially unmalted.

3. In some embodiments of the process of paragraph 1 or 2, the coarsely ground grains or seeds decrease cake resistance of insoluble matter produced during steeping compared to an equivalent process using more finely-ground grains or seeds used in a conventional dry grind milling process.

4. In some embodiments of the process of any of the preceding paragraphs, the starch hydrolysate is separated from depleted coarsely-ground grains or seeds during or following recovery of the starch hydrolysate from the steeping water.

5. In some embodiments of the process of paragraph 4, the starch hydrolysate and starch-depleted coarsely ground grains or seeds are separated by filtration.

6. In some embodiments of the process of paragraph 5, filtration is by way of filtration using starch-depleted coarsely ground grain or seeds as a filtration matrix in the same or different vessel used for steeping.

7. In some embodiments of the process of any of the preceding paragraphs, the thermostable α-amylase is exogenous or non-native with respect to the grains or seeds.

8. In some embodiments of the process of paragraph 7, the exogenous or non-native thermostable α-amylase is added to the steep water in the form of an enzyme formulation or whole cell broth fermentation product.

9. In some embodiments of the process of paragraph 7 or 8, at least a portion of the thermostable α-amylase is added to the steep water prior to contacting the steep water with the coarsely ground grains or seeds.

10. In some embodiments of the process of the process paragraph 7, the exogenous or non-native thermostable α-amylase is produced by genetically modified grains or seeds.

11. In some embodiments of the process of any of the preceding paragraphs, steeping is performed in the presence of a secondary exogenous or non-native beneficial enzyme.

12. In some embodiments of the process of paragraph 11, the secondary exogenous or non-native enzyme is a carbohydratase, protease, cellulase, xylanase or phytase.

13. In some embodiments of the process of paragraph 12, the secondary exogenous or non-native enzyme is a glucoamylase and the starch hydrolysate includes an increased amount of glucose.

14. In some embodiments of the process of the preceding paragraphs, the average particle size of the coarsely-ground grains or seeds is greater than at least 50%, at least 60%, at least 70%, at least 70%, at least 80%, at least 90% or greater, than particles screened though a 500 μm filter.

15. In some embodiments of the process of any of the preceding paragraphs, the average particle size of the coarsely-ground grains or seeds is greater than 2 mm.

16. In some embodiments of the process of any of the preceding paragraphs, the steeping temperature is at least 75° C.

17. In some embodiments of the process of any of the preceding paragraphs, steeping is performed without subsequent germ separation.

18. In some embodiments of the process of any of the preceding paragraphs, the soluble starch hydrolysate is fermented.

19. In some embodiments of the process of paragraph 18, fermentation is performed without prior degerming.

20. In some embodiments of the process of paragraph 18 or 19, fermentation is performed without prior centrifugation.

21. In some embodiments of the process of any of the preceding paragraphs, fermentation is performed without prior or subsequent fiber separation.

22. An ethanol fermentation process utilizing the steep liquor described in previous paragraphs, where the ethanol concentration exceeds 15% w/w in the final fermentation broth.

These and other aspects and embodiments of the present compositions and processes/methods will be apparent from the following description and appended Examples.

### BRIEF DESCRIPTION OF THE DRAWINGS

FIG. 1 is a graph showing the mean particle size of differentially ground corn.

FIG. 2 shows the extraction of dissolved solids, in the presence of a thermostable α-amylase, from differentially ground corn over time.

FIG. 3 is a graph showing permeability coefficient versus mean particle size for steeping experiments using different grinds.

### DETAILED DESCRIPTION

### 1. Definitions and Abbreviations

In accordance with this detailed description, the following abbreviations and definitions apply. Note that the singular forms “a,” “an,” and “the” include plural referents unless the context clearly dictates otherwise. Thus, for example, reference to “an enzyme” includes a plurality of such enzymes, and reference to “the enzyme” includes reference to one or more enzymes and equivalents thereof known to those skilled in the art, and so forth.

The present document is organized into a number of sections for ease of reading; however, the reader will appreciate that statements made in one section may apply to other sections. In this manner, the headings used for different sections of the disclosure should not be construed as limiting.

Unless defined otherwise, all technical and scientific terms used herein have the same meaning as commonly understood by one of ordinary skill in the art. The following abbreviations and terms are defined, below, for clarity.

### 1.1. Abbreviations and Acronyms

The following abbreviations/acronyms have the following meanings unless otherwise specified:
° C. degrees centigrade
CP centipoise
DS dissolved solids
g or gm grams
hr hour
gpm gallons per minute
kg kilograms
m meters
mg milligrams
min minute
mL and ml milliliters
mm millimeters
Pa pascals
Pa·s pascal seconds (1,000 cP)
rpm revolutions per minute
S seconds
U units
v/v volume/volume
w/v weight/volume
w/w weight/weight

Particular abbreviations/acronyms associated with Darcy's law have the following meanings
q liquid flux in m/s
u dynamic viscosity in Pa
length of the sample in m L
dP pressure drop in Pa
k permeability coefficient in m²

### 1.2. Definitions

The term “starch” refers to any material composed of the complex polysaccharide carbohydrates of plants, comprised of amylose and/or amylopectin with the formula (C6H1005) x, wherein X can be any number. In particular, the term refers to any plant-based material including but not limited to grains, grasses, tubers and roots and more specifically wheat, barley, corn, rye, rice, sorghum, legumes, cassava, millet, potato, sweet potato, and tapioca. After purification of the complex polysaccharide carbohydrates from the other plant components, it is called “refined starch.”

“Soluble starch hydrolysates” are polysaccharides derived from insoluble starch that are soluble in water at room temperature or greater. Soluble starch hydrolysates include dextrins and malto-oligosaccharides but are low in glucose content.

“Dextrins” are linear and partially branched soluble and insoluble polysaccharides produced by the partial hydrolysis of insoluble starch.

“Malto-oligosaccharides” are linear and partially branched polysaccharides produced by the hydrolysis of insoluble starch and dextrins by acid treatment or by treatment with endo-acting carbohydrases such as α-amylase amylases.

“Maltodexrins” refers to refined malto-oligosaccharides, generally ranging from DP3 to DP20, but can be longer.

“Hydrolysed starch syrups” are polysaccharides derived from insoluble starch or soluble starch hydrolysates that are rich in short malto-oligosaccharides, e.g., DP5 and less, including glucose.

The term “α-amylase” refers to an enzyme that is, among other things, capable of catalyzing the degradation of starch. α-amylases are hydrolases that cleave the α-D-(1→4) O-glucosidic linkages in starch. Generally, α-amylases (EC 3.2.1.1; α-D-(1→4)-glucan glucanohydrolase) are defined as endo-acting enzymes cleaving α-D-(1→4) O-glucosidic linkages within the starch molecule in a random fashion yielding polysaccharides containing three or more (1-4)-α-linked D-glucose units.

The terms “thermostable” and “thermostability,” with reference to an enzyme, refer to the ability of the enzyme to retain activity after exposure to an elevated temperature. The thermostability of an enzyme, is measured by its half-life (t_{1/2}) given in minutes, hours, or days, during which half the enzyme activity is lost under defined temperature conditions. Half-life may be calculated by measuring residual activity following exposure to the elevated temperature. Thermostability may also be estimated based on the temperature optimum of the enzyme.

A “pH range,” with reference to an enzyme, refers to the range of pH values under which the enzyme exhibits catalytic activity.

The term “endogenous” with reference to a polynucleotide or protein refers to a polynucleotide or protein that occurs naturally in the host cell, and is expressed by that host cell, in a given situation.

The term “exogenous” and “non-native” with reference to a polynucleotide or protein refers to a polynucleotide or protein that may or not occur naturally in the host cell, but is added as an enzyme solution or suspension, without the need for viable host cells.

The term “native” with reference to a polynucleotide or protein refers to a polynucleotide or protein that occurs naturally in the host cell, but is either is expressed by that host cell, or added as an enzyme solution or suspension, without the need for viable host cells.

The term “non-native” with reference to a polynucleotide or protein refers to a polynucleotide or protein that does not occur naturally in the host cell, but is either is expressed by that host cell, or added as an enzyme solution or suspension, without the need for viable host cells.

The terms “recovered,” “isolated,” and “separated,” refer to a compound, protein (polypeptides), cell, nucleic acid, amino acid, or other specified material or component that is removed from at least one other material or component with which it is naturally associated as found in nature. An “isolated” polypeptides, thereof includes, but is not limited to, a culture broth containing secreted polypeptide expressed in a heterologous host cell.

The term “purified” refers to material (e.g., an isolated polypeptide or polynucleotide) that is in a relatively pure state, e.g., at least about 90% pure, at least about 95% pure, at least about 98% pure, or even at least about 99% pure.

The term “enriched” refers to material (e.g., an isolated polypeptide or polynucleotide) that is in about 50% pure, at least about 60% pure, at least about 70% pure, or even at least about 70% pure.

The phrase “simultaneous saccharification and fermentation,” or “SSF,” refers to a process in the production of biochemicals in which a microbial organism, such as an ethanologenic microorganism, and at least one enzyme, such as an amylase, are present during the same process step. SSF includes the contemporaneous hydrolysis of starch substrates (granular, liquefied, or solubilized) to saccharides, including glucose, and the fermentation of the saccharides into alcohol or other biochemical or biomaterial in the same reactor vessel.

The phrase “substantially unmalted” refers to grains or cereal that have not been allowed to germinate at all, or to an extent where germination affects the performance or purpose of the present sweet steep process.

The term “about” refers to #15% to the referenced value.

### 2. Process for Making Clean Sugar

Applicants recently described a process for obtaining commercially valuable products from grains or seeds that required fewer processing steps and less energy than conventional wet milling (WO2021195541). It also produces higher quality animal feed products, more oil, and uses less energy, compared to conventional dry-grind milling. The process involved steeping substantially intact grains or seeds at a temperature at or above the starch gelatinization temperature in the presence of a thermostable α-amylase. The process was dubbed “corn sweet steeping,” to reflect the presence of fermentable starch hydrolysates in the steeping water, resulting from the action of the thermostable α-amylase.

Applicants have now improved the sweet steeping process by course-grinding the corn kernels prior to steeping to increase the rate of conversion of starch by the thermostable α-amylase, while avoiding filtration difficulties resulting from excess caking.

A key to the improved process is the nominal size of the of the milled grain or seed particles subjected to elevated temperature steeping. While some milling is required to disrupt intact grains or seeds, finer grinding, e.g., as used for conventional dry milling, presents other challenges. For sweet steeping to be maximally efficient, the starch hydrolysate-containing steep water produced by the thermostable α-amylase (i.e., sweet steep water) should be depth filterable, to enable the economic recovery of a clean starch hydrolysate stream from the steeping process. Such depth filtration uses the starch-depleted grain or seed particles as the filter medium, making the size of the particles a critical factor.

Conventional dry grind particles produce product sweet steep water that with high cake resistance, which is not suitable for producing a post sweet steep filtration medium suitable for depth filtration. Conventional dry grind particles are typically in the range of a mean particle size of 800 to 1,000 μm, while the preferred mean particle size for the improved sweet steep process is in the range of 2 to 4 mm. Accordingly, while no milling is an option for starch hydrolysate production using the original sweet steep process, and conventional dry milling grind is not well suited to sweet steeping, the use of course-ground grain or seeds in combination with sweet steeping has the potential to improve the economics of ethanol production.

Suitable coarsely-ground grains or seeds have a mean diameter greater than 2 mm. By comparison, more finely-ground grains or seeds are used in conventional dry grind milling processes. Coarsely ground grains or seeds with a mean diameter greater than 2 mm have a lower resistance to liquid flow through a packed bed. This enables liquid recirculation with minimal (or the absence of) mechanical mixing, and enables effective washing using a lauter-like process. An additional benefit is the germ is subject to less fracturing as particle size increases, reducing the amount of germ associated oil released into the liquid fraction.

As with the originally described sweet steep process, the improved process can be performed in the absence of SO₂ and lactic acid, which are used in conventional steeping processes to cause grains to soften, and to weaken the gluten-starch matrix, allowing for eventual separating of starch granules for other parts of the grain. Such starch separation from other grain materials subsequently occurs by centrifugation, requiring that the starch material remain in a dense form to affect separation. No centrifugation is required in the sweet steep process or the improved process for producing starch hydrolysates.

A preferred method is to introduce enzyme to liquor before corn is subject to steeping liquid near the gelatinization temperature. This can be performed by adding thermostable α-amylase to corn slurry prior to (i.e., below) a temperature near gelling, for example, 50° C. or so. Alternatively, enzyme can be added to hot steeping liquor if that liquor is then batched into dry corn. A key feature of the present methods is that the thermostable α-amylase must be present in the corn slurry before there is significant water migration into the grain.

Also as with the originally described sweet steep process, steeping is performed at or above the starch gelatinization of, processes/methods, corn at a temperature of at least about 68° C., at least about 70° C., at least about 72° C., at least about 74° C., at least about 76° C., at least about 78° C., at least about 80° C., at least about 82° C., at least about 84° C., at least about 86° C., at least about 88° C. or even at least about 90° C., coordinated with contacting corn kernels with water containing thermostable α-amylase. Exemplary steeping temperatures ranges are about 70-100° C. and 80-90° C. The elevated temperatures deactivate endogenous starch hydrolyzing enzymes, ensuring that uncontrolled starch hydrolysis is avoided and that the starch hydrolysis pattern is determined by the recombinant or exogenous/non-native thermostable α-amylase(s) selected for using in steeping.

The elevated temperature also deactivates endogenous proteases, leaving course-ground grain or seed proteins intact and minimizing uncontrolled hydrolysis of corn proteins to peptides or free amino acids. This avoids the loss of peptides and amino acids in the steeping water, and maintaining the protein content of the grain, and further without disrupting the germ.

### 3. Clean Sugar Recovery Process

At the end of the original sweet steeping process, mild pressing, centrifugation or other mild mechanical means could optionally be performed to extract remaining soluble starch hydrolysates from the starch-depleted solids/particles. Mild mechanical means referred to those that would cause soluble starch hydrolysates to be separated from the grain or seed residuals but would not otherwise fractionate or disrupt the corn or seed residual. Such additional mechanical processing is likely less important when using course-ground grains or seeds because the grain or seed is no longer intact; however, it remains an option for improving yield.

Starch hydrolysates in the steeping water can be efficiently recovered by way of depth filtration, using the starch hydrolysate-depleted course-ground grain or seed particles as a filter medium. In this manner, a starch hydrolysate stream for fermentation is produced in a manner analogous to the process of lautering used in brewing. The starch hydrolysates may be recycled through the filter medium to improve clarity and a final rinse of the filter medium can be used to maximize recovery. The clean starch hydrolysate stream produced by the improved sweet steep process can be used as fermentable starch hydrolysate for making ethanol or other valuable fermentation products.

As with the original sweet steep process, the improved process does not rely on the fractionation of the grain or seeds prior to steeping, such as degerming in the case of dry fractionation. Additionally, the improved process does not rely on the fractionation of steeped grain or seeds prior to fermentation, as in the case of germ, fiber or oil separation, in wet milling. Nor does the improved process require mechanical processing of a saccharification product prior to fermentation.

The starch-depleted course ground grain of seeds represents high quality animal feed and otherwise trapped oil. The oil can be part of the animal feed or separated for other uses, including the production biodiesel. Such side-products of the sweet steep process are not fermentation residuals, are not stillage products, and have not been subject to distillation.

### 4. Thermostable α-Amylases for Use in Modified Sweet Steeping

Wet and dry-grind milling using thermostable α-amylases has been described. These enzymes include commercially available bacterial enzymes, such as SPEZYME®-AA, SPEZYME®-Alpha, SPEZYME®-Ethyl, SPEZYME®-Fred, SPEZYME®-Xtra and SPEZYMER-RSL, CLARASE™ L, GZYME™ 997 and GC356 (DuPont), TERMAMYL™ 120-L, TERMAMYL™ LC and TERMAMYL™ SC and SUPRA, LIQUOZYME™ X, SAN™ SUPER, LPHERA® and FORTIVA® (Novozymes A/S), and FUELZYME™ LF (Diversa). Commercially available thermostable fungal enzymes include GC626® (DuPont) from Aspergillus kawachii.

Preferred thermostable α-amylases have optimal activity (for ground corn) of at least about 68° C., at least about 70° C., at least about 72° C., at least about 74° C., at least about 76° C., at least about 78° C., at least about 80° C., at least about 82° C., at least about 84° C., at least about 86° C., at least about 88° C. or even at least about 90° C.

### 5. Glucoamylases for Use in Modified Sweet Steeping

Glucoamylases for use in elevated temperature steeping include but are not limited to those described for use in wet and dry-grind milling. Commercially available thermostable glucoamylase enzymes include EXTENDA® and SPIRIZYME® (Novozymes). In some embodiments, the thermostable glucoamylase will be derived from an organism such as a Talaromyces sp., Clostridium sp. or a Penicillium sp.

Commercially available thermostable phytase enzymes include AXTRA® PHY (DuPont) and RONOZYME® (Novozymes) and FUELZYME® from BASF. In some embodiments, the thermostable phytase will be derived from an organism such as Buttiauxella sp., a Citrobacter sp, an Escherichia sp., a Peniophora sp. or an Obesumbacterium sp.

Commercially available thermostable protease enzymes include DCO+® (DuPont) and AVENTEC® AMP (Novozymes). In some embodiments, the thermostable protease will be derived from an organism such as a Thermobifida sp., a Nocardiopsis sp., a Thermococcus sp. a Streptomyces sp. or a Pyrococcus sp. A classic thermostable protease is thermolysin, a neutral metalloproteinase produced by the Gram-positive bacteria Bacillus thermoproteolyticus.

The addition of glucoamylase (and other carbohydrate processing enzymes) in the sweet steep process will result in high concentrations of glucose in the starch hydrolysate stream, which may improve the initial rate of fermentation, especially where most of the glucoamylase in fermentation is provided by glucoamylase-expressing yeast. In fact, such addition of glucoamylase to the steeping process may allow all the glucoamylase in fermentation to be produced by glucoamylase-expressing yeast.

Glucoamylases should be sufficiently thermostable to withstand elevated steeping temperatures. Alternatively, they may be added later in the steeping process, such that they are not required to survive the entire steep duration, and/or the most elevated steep temperatures. Glucoamylases may instead or additionally be added to soluble starch hydrolysates or hydrolyzed starch syrups fractions after the sweet-steepened liquor (i.e., steep liquor subject to the steeping process) cools, in which cases they may not have to be thermostable.

### 6. Additional Enzymes for Use During Sweet Steeping

In some variations of the new processes additional enzymes can be added during steeping to enhance the production of starch hydrolysates or, change the profile of the starch hydrolysates or to reduce the anti-nutritional factors, such as phytic acid. Such enzymes include pullulanase, β-amylase, maltogenic α-amylase isoamylase, trehalase, phytase and the like. In addition, non-starch hydrolyzing enzymes such as cellulase, glucanase, xylanase, pectinase, protease and phytase can also be included. Enzymes for use in elevated temperature steeping include but are not limited to those described for use in wet and dry-grind milling.

In the case of protease, addition in the sweet steep process will reduce the amount of intact protein in the starch-depleted course-ground grain or seeds but may be desirable to produce amino acids for the yeast growth during optional fermentation, which would reduce the need supplementation with an external nitrogen source. Residual amino acids would be recovered in the clean strain of starch hydrolysates.

Protease and/or other additional enzymes should be sufficiently thermostable to withstand elevated steeping temperatures. Alternatively, these enzymes may be added later in the steeping process, such that they are not required to survive the entire steep duration and/or the most elevated steep temperatures. Such enzymes may instead or additionally be added to soluble starch hydrolysates or hydrolyzed starch syrups fractions after the sweet-steepened liquor cools, in which cases they may not have to be thermostable.

The addition of protease in the sweet steep process will reduce the amount of intact protein in the starch-depleted course-ground grain or seeds but may be desirable to produce amino acids for the yeast growth during optional fermentation, which would reduce the need supplementation with an external nitrogen source. Residual amino acids would be recovered in the clean strain of starch hydrolysates.

### 7. Inactivation of Endogenous Enzymes During Sweet Steeping

An advantage of modified sweet steeping, is the inactivation of endogenous enzymes, including proteases, leaving proteins intact for feeding to animals or preparing foodstuffs. However, in some variations of the new process, endogenous proteases are not entirely inactivated, or selected proteases, including thermostable proteases, are deliberately added in amount to aid release of starch from the course-ground grain of seed, to supplement the clean starch hydrolysate with amino acids, as mentioned above.

### 8. Enzymes Preparations

Thermostable α-amylases, as well as other enzymes used for the modified sweet steep process, may be in the form of purified, concentrated and/or formulated enzyme preparations, or in the form of whole cell broth or clarified whole cell broth preparations. Suitable preparations are those described for wet and dry grind milling. Enzyme preparations may include any number of additional beneficial components.

All references cited herein are herein incorporated by reference in their entirety for all purposes.

### EXAMPLES

### Example 1. Production of Ground Corn

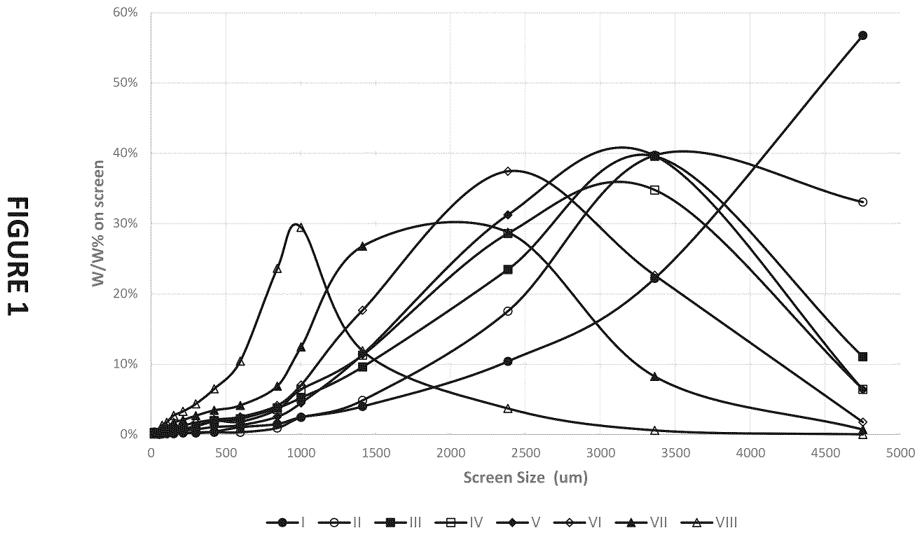

Several different batches of ground corn were supplied by CPM Milling (Iowa, USA). The mean particle size of each batch was determined by sieving and is shown in FIG. 1. Batches I-VII represent corn that is increasingly finely milled. Batch I included intact kernels and Batch VII is equivalent to the ground corn used in a typical dry grind process. Batch VIII represents minimally milled, substantially intact kernels (which may for convenience be referred to as “intact” and/or “whole”). Batches I, II, III, V and VIII were further analyzed for protein, fat, fiber, ash and moisture content. The results (in w/w %) are summarized in Table 1. Mean particle size (in μm) is also reported. Mean particle size for whole corn was based on published data.

TABLE 1
Composition of steep liquor resulting
from different batches of ground corn

| | Mean | | | | | |
|---|---|---|---|---|---|---|
| Batch | particle size | Protein | Fat | Fiber | Ash | Moisture |
| I | 7000 | 0.15 | 0.00 | 0.00 | 0.10 | 93.44 |
| II | 3755 | 0.19 | 0.00 | 0.00 | 0.11 | 90.97 |
| III | 2705 | 0.20 | 0.00 | 0.00 | 0.12 | 90.66 |
| V | 2183 | 0.23 | 0.00 | 0.00 | 0.12 | 89.51 |
| VIII | 930 | 0.24 | 0.07 | 0.01 | 0.14 | 87.22 |

### Example 2. Starch Hydrolysate Extraction Rate of Ground Corn

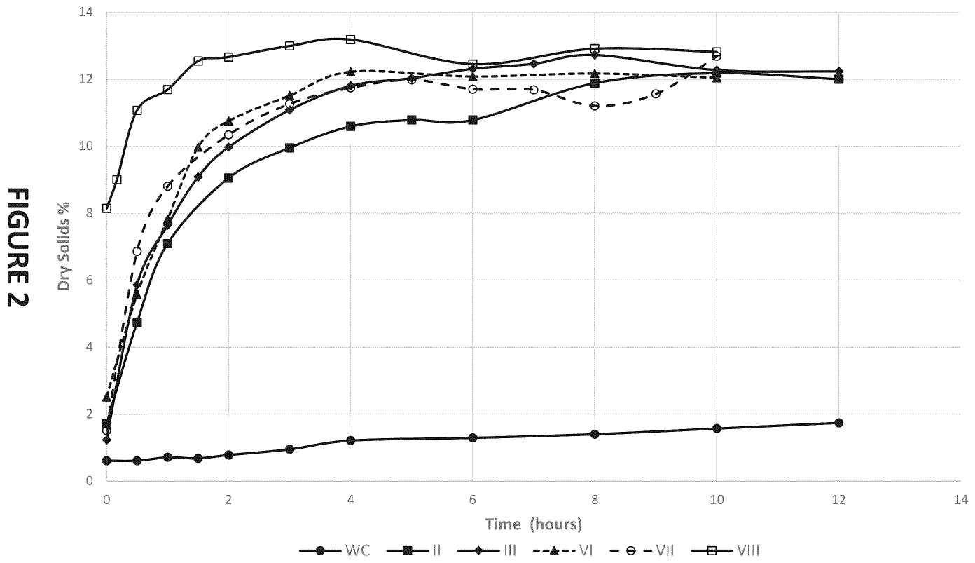

Portion of Batches I, II, V, VI, VII and whole corn (equivalent to Batch VIII, above) were incubated in the presence of 0.3 U/g thermostable α-amylase (SPEZYME® HT; IFF), which is about 1.6 times a typical dry grind dose. The w/w % DS was measured over a course of 12 hr. The results are shown in FIG. 2. The finer the grind, the faster the DS increase; however, all batches except whole corn eventually reached the same end point over the duration of the experiment.

### Example 3. Cake Resistance of Ground Corn

Six-each sample of batched milled corn were treated as follows: 500 g of corn and 1,500 g of water were added to 2-L roller bottles and well mixed. The roller bottles were placed into an incubator on an orbital plate at 80° C. and 120 rpm. Following a 1-hr incubation, the bottles were checked for pH to make sure they were within the range of pH 5.3-6.0. 1.5 ml of 10% CaCl₂) was added to each sample to a final concentration of 100 ppm. A thermostable (and thermophilic) α-amylase was added to each sample to a final dose of 0.35 U/g-dry solids. The bottles were returned to the same incubator, allowed to steep for 5 hr, and then removed from the incubator and the liquid drained. To each bottle 1,000 ml of warm water was added, mixed and allowed to stand for 15 min. The rinse water was drained, and the wash was repeated. The residual corn was harvested, packaged and refrigerated in preparation for shipping to a cake resistance test site.

The cake resistance of each steeped and washed batch was measured and is reported in Table 2.

TABLE 2
Cake resistance of steeped and washed ground corn

| Batch | mean particle size (μm) | Flow Rate (g/s) |
|---|---|---|
| I | 4053 | 52.5 |
| II | 3755 | 49.7 |
| III | 2705 | 46.6 |
| VI | 2183 | 49.5 |
| VII | 1607 | 43.0 |
| Whole | 930 | 29.2 |

### Example 4. Characterization of Different Grades of Coarsely Milled Corn

### Milling

Three tons of USDA yellow dent #2 corn was acquired for the purpose of generating different grades of coarsely milled corn. Different mean particle size samples were generated by adjusting the gaps between the rollers.

Five different grinds of corn (i.e., Grinds 1-5) were produced and subjected to particle size analysis using USA standard mesh sieves. Sieved weight percent is summarized in Table 3. Calculated bulk properties of the different grinds of corn, based on sieved weight percent and moisture content of 12.85%, are summarized in Table 4.

TABLE 3
Sieved properties of samples

| | Opening | | | | | |
|---|---|---|---|---|---|---|
| US mesh | (μm) | Grind 1 | Grind 2 | Grind 3 | Grind 4 | Grind 5 |
| 4 | 4749 | 0.00 | 0.19 | 1.35 | 8.53 | 38.73 |
| 6 | 3360 | 0.06 | 5.41 | 23.56 | 40.64 | 37.91 |
| 8 | 2380 | 1.38 | 30.77 | 36.84 | 25.85 | 12.62 |
| 12 | 1410 | 9.19 | 29.18 | 15.26 | 9.69 | 4.19 |
| 16 | 1000 | 36.63 | 13.52 | 7.45 | 5.35 | 2.50 |
| 20 | 841 | 22.20 | 6.27 | 4.22 | 3.03 | 1.30 |
| 30 | 594 | 11.13 | 4.12 | 3.19 | 2.17 | 0.92 |
| 40 | 420 | 6.54 | 2.85 | 2.31 | 1.46 | 0.63 |
| 50 | 297 | 3.11 | 0.90 | 0.77 | 0.50 | 0.24 |
| 70 | 212 | 2.25 | 0.80 | 0.70 | 0.40 | 0.14 |
| 100 | 150 | 2.59 | 1.05 | 0.85 | 0.56 | 0.19 |
| 140 | 103 | 1.33 | 0.53 | 0.42 | 0.30 | 0.10 |
| 200 | 73 | 0.98 | 0.47 | 0.36 | 0.30 | 0.05 |
| 270 | 53 | 0.58 | 0.32 | 0.16 | 0.20 | 0.05 |
| Pan | | 2.03 | 3.62 | 2.56 | 1.01 | 0.90 |

TABLE 4
Bulk properties of samples

| Property | Grind 1 | Grind 2 | Grind 3 | Grind 4 | Grind 5 |
|---|---|---|---|---|---|
| Mean particle | 888 | 1537 | 2020 | 2656 | 3791 |
| size (μm) | | | | | |

### Steeping

Samples of each grind as well as a sample of whole corn was steeped. 125 kg of water was added to a jacketed 100-gallon scrape kettle utilizing a side scaping blade along with a central blending mixer. 25 kg corn was added with mixing and 125 ml of 10% CaCl₂) was added to the slurry. The mixture was heated to 50° C. and adjusted to pH 5.5 with H₂SO₄. 21.5 ml of SPEZYME® HT (a commercially available, high-temperature α-amylase at a concentration of 350 U/g) was added to achieve a steeping dry solids dose of 0.3 U/g. Final steeping temperature was ramped up to 85° C., with steeping time being recorded once the temperature reached 72° C.

Samples were taken at 0, 0.5, 1, 2, 3 and 4 hr, and dry solids were measured. After 4 hr steeping, the steep slurry was cooled to about 50° C. and harvested. Liquid-solid separation was performed on a vibrating SWECO sieve (300 μm) and the weights and moisture content of the different fractions were measured. Dry weight percent is summarized in Table 5.

TABLE 5
Dry weight percent of steeped samples

| | Dry weight percent | | | | | |
|---|---|---|---|---|---|---|
| Time | Grind 1 | Grind 2 | Grind 3 | Grind 4 | Grind 5 | Whole |
| 0 | | 7.22 | 4.50 | 2.94 | 2.00 | 0.60 |
| 0.5 | 11.45 | 9.91 | 7.49 | 7.06 | 5.87 | 0.40 |
| 1 | 12.21 | 10.73 | 8.84 | 9.24 | 9.68 | 1.08 |
| 2 | 12.60 | 12.14 | 10.83 | 10.60 | 8.86 | 1.73 |
| 3 | 13.39 | 13.34 | 12.05 | 11.16 | 9.88 | 2.87 |
| 4 | 13.34 | 13.77 | 12.37 | 11.96 | 10.82 | 4.01 |
| 5 | | | | | | 5.27 |

### Example 5. Characterization of Steep Liquors Obtained Using Different Grinds

Steep liquor from the steeping trial in Example 4 was put into a “mash n boil” tank and set to 85° C. (185° F.). Steeped ground corn was loaded into a polycarbonate column while the emergent liquor was heating. The column was filled with liquid up to the overflow port. Once the system was stabilized at the set temperature, liquid flow was collected and filtered through a 420-μm bottom-screen. Liquid viscosity and density were measured so that the permeability coefficient could be calculated using Darcy's law:

k=quL/dP
where: q denotes the liquid flux in m/s
   u denotes the dynamic viscosity in Pa's
   L denotes the length of the sample in m
   dP denotes the pressure drop in Pa
   k is the permeability coefficient in m²

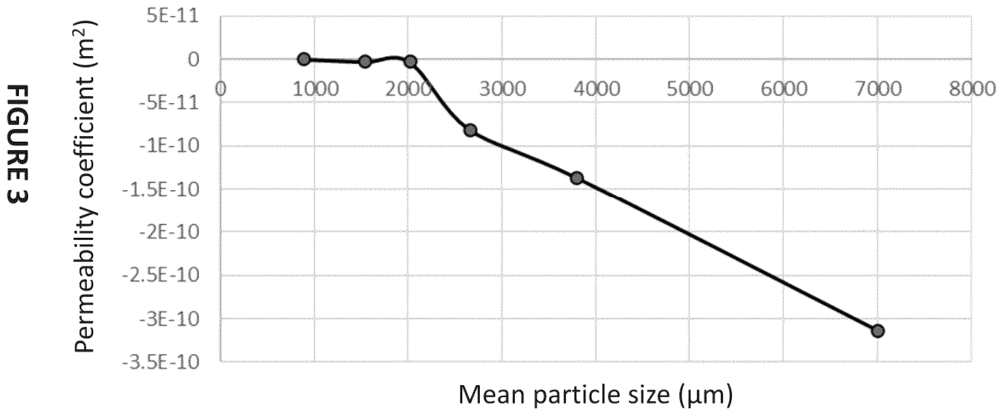

The flow rate and other system parameters are summarized in Table 6. The 7,000 μm size value for sample for intact whole corn (Grind VII) is an assumed value based on literature. The graph in FIG. 3 shows the abrupt transition in flowability below 2,000 μm.

TABLE 6
Steeping trial flow test data and calculations

| | Mean | | Bed | Liq. | Flow | |
|---|---|---|---|---|---|---|
| | size | Diameter | height | height | rate | Perm. coef. |
| Sample | (μm) | (in) | (in) | (in) | (gpm) | (m²) |
| Grind 1 | 888 | 5.75 | 6 | 24.5 | 3 | −8.36 × 10⁻¹⁴ |
| Grind 2 | 1537 | 5.75 | 6 | 24.5 | 109 | −3.01 × 10⁻¹² |
| Grind 3 | 2020 | 5.75 | 6 | 24 | 115 | −3.28 × 10⁻¹² |
| Grind 4 | 2656 | 5.75 | 12 | 24 | 1425 | −8.14 × 10⁻¹¹ |
| Grind 5 | 3791 | 5.75 | 12 | 24 | 2400 | −1.37 × 10⁻¹⁰ |
| Whole | 7000 | 5.75 | 12 | 24 | 5440 | −3.14 × 10⁻¹⁰ |

### Example 6. Composition of Residual Solids of Steeped Coarse Ground Corn

Samples of the solids remaining after removal of the steep liquor from the process described in Example 4 were submitted for proximate analysis to determine the retention of the major constituents in corn. A summary of the analysis is presented in Table 7. Numbers are in percent.

TABLE 7
Component retention in residual corn solids

| Sample | Protein | Crude Fat | Crude fiber | Ash | Starch |
|---|---|---|---|---|---|
| Grind 4 | 90.4 | 99.1 | 83.5 | 60.5 | 2.2 |
| Grind 5 | 85.8 | 101.6 | 122.1 | 63.7 | 5.1 |

What is claimed is:

1. A process for producing a soluble starch hydrolysate from grains or seeds, comprising steeping coarsely-ground grains or seeds at a temperature above the gelatinization temperature of starch in the grains or seeds in the presence of a thermostable α-amylase to produce a starch hydrolysate from starch present in the grains or seeds; and recovering the soluble starch hydrolysate from the steeping water.

2. The process of claim 1, wherein coarsely ground grains or seeds are substantially unmalted.

3. The process of claim 1 or 2, wherein the coarsely ground grains or seeds decrease cake resistance of insoluble matter produced during steeping compared to an equivalent process using more finely-ground grains or seeds used in a conventional dry grind milling process.

4. The process of any of the preceding claims, wherein the starch hydrolysate is separated from depleted coarsely-ground grains or seeds during or following recovery of the starch hydrolysate from the steeping water.

5. The process of any of claim 4, wherein the starch hydrolysate and starch-depleted coarsely ground grains or seeds are separated by filtration.

6. The process of claim 5, wherein filtration is by way of filtration using starch-depleted coarsely ground grain or seeds as a filtration matrix in the same or different vessel used for steeping.

7. The process of any of the preceding claims, wherein the thermostable α-amylase is exogenous or non-native with respect to the grains or seeds.

8. The process of claim 7, wherein the exogenous or non-native thermostable α-amylase is added to the steep water in the form of an enzyme formulation or whole cell broth fermentation product.

9. The process of claim 7 or 8, wherein at least a portion of the thermostable α-amylase is added to the steep water prior to contacting the steep water with the coarsely ground grains or seeds.

10. The process of claim 7, wherein the thermostable α-amylase is produced by genitically-modified grains or seeds.

11. The process of any of the preceding claims, wherein steeping is performed in the presence of a secondary exogenous or non-native beneficial enzyme.

12. The process of claim 11, wherein the secondary exogenous or non-native enzyme is a carbohydratase, protease, cellulase, xylanse or phytase.

13. The process of claim 12, wherein the secondary exogenous or non-native enzyme is a glucoamylase and the starch hydrolysate includes an increased amount of glucose.

14. The process of any of the preceding claims, wherein the average particle size of the coarsely-ground grains or seeds is greater than at least 50%, at least 60%, at least 70%, at least 70%, at least 80%, at least 90% or greater, than particles screened though a 500 μm filter.

15. The process of any of the preceding claims, wherein the average particle size of the coarsely-ground grains or seeds is greater than 2 mm.

16. The process of any of the preceding claims, wherein the steeping temperature is at least 75° C.

17. The process of any of the preceding claims, wherein steeping is performed without subsequent germ separation.

18. The process of any of the preceding claims, wherein the soluble starch hydrolysate is fermented.

19. The process of claim 18, wherein fermentation is performed without prior degerming.

20. The process of claim 18 or 19, wherein fermentation is performed without prior centrifugation.

21. The process of any of the preceding claims, wherein fermentation is performed without prior or subsequent fiber separation.

22. An ethanol fermentation process utilizing the steep liquor described in previous claims, where the ethanol concentration exceeds 15% w/w in final fermentation broth.
